(19) United States
(12) Patent Application Publication
(10) Pub. No.: US 2010/0222606 A1
(43) Pub. Date: Sep. 2, 2010

(54) METHOD FOR PRODUCING BISABOLOL WHICH IS FARNESOL FREE OR IS LOW IN FARNESOL

(75) Inventors: Hansgeorg Ernst, Speyer (DE); Klaus-Peter Pfaff, Friedelsheim (DE); Karl Beck, Ostringen (DE); Jürgen Schubert, Dirmstein (DE); Günther Gottwald, Mannheim (DE); Wolfgang Krause, Bruhl-Rohrhof (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/160,918

(22) PCT Filed: Jan. 12, 2007
(86) PCT No.: PCT/EP2007/050297

§ 371 Date: Jul. 15, 2008

(30) Foreign Application Priority Data
Jan. 16, 2006 (EP) 06100395.0

(51) Int. Cl. C07C 35/18; C11C 1/00

(52) U.S. Cl. 554/163; 568/826

(57) ABSTRACT

The present invention relates to a method of producing pure or enriched bisabolol by separating substance mixtures comprising bisabolol and farnesol by selective esterification of farnesol and subsequent distillative separation. The invention relates specifically to a method as specified above comprising the selective transesterification of mixtures comprising formyl-bisabolol and formyl-farnesol and subsequent distillative separation.

The present invention furthermore relates to a method of producing farnesol esters.

### TECHNICAL FIELD OF THE INVENTION

The present invention relates to a method of producing farnesol-free or low-farnesol bisabolol by separating substance mixtures comprising bisabolol and farnesol by selective esterification of farnesol and subsequent distillative separation. The invention relates specifically to a method as specified above comprising the selective transesterification of mixtures comprising formyl-bisabolol and formyl-farnesol and subsequent distillative separation. The present invention furthermore relates to a method of producing farnesol esters.

alpha-Bisabolol is one of the most important constituents of camomile oil, which is valuable from a cosmetics and pharmaceutical point of view. It is a sought-after active ingredient which is used in creams, ointments and lotions for skin protection and skin care. Moreover, it is used in sunscreen preparations, aftersun cosmetics, infant care compositions, aftershave products and oral care preparations.

While the systematic cultivation of medicinal plants continues to gain importance on account of an increased demand for “renewable raw materials” and for natural active ingredients, the limited natural resources have at the same time led to the search and development of methods of obtaining synthetic products.

Synthetic “alpha-bisabolol” is usually a diastereomeric racemate of equal parts (+/−)-α-bisabolol and (+/−)-epi-α-bisabolol. All four enantiomers have been found in nature.

On account of its described effects, there is a constant need for (+)-, (−)- and (+/−)-alpha-bisabolol, and/or (+)-epi- , (−)-epi- and (+/−)-epi-α-bisabolol, i.e. for compounds of the formula (Ia)

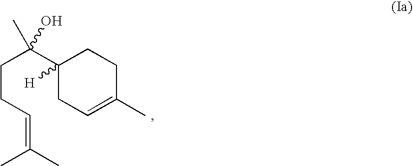

in which wavy lines are in each case independently of one another an S or R configuration on the appertaining carbon atom. For example, a large number of methods and processes for producing bisabolol starting from nerolidol have been described in the past.

The racemic mixture of I- and d-alpha-bisabolol, used primarily in cosmetics, is often produced industrially by acid-catalyzed cyclization of farnesol of the formula (II)

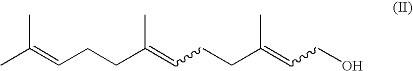

and nerolidol of the formula (VII)

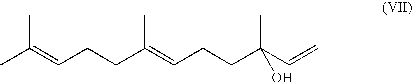

as described, inter alia, also in DE 102 46 038.

A catalyst which is often used for the cyclization of said compounds of the formulae (II) or (VII) to give alpha-bisabolol of the formula (I) is formic acid. Due to the better conversion and the higher reaction rate, nerolidol is better suited for this process, as described in Tetrahedron 24, 859 f. The main product obtained here is alpha-bisabolol formate of the formula (V)

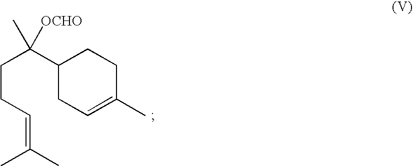

as by-product, farnesol formate of the formula (VI) arises as a result of allyl rearrangement

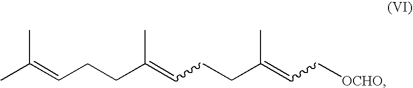

where the wavy lines in this case refer to isomers with regard to the configuration of the respective ethylenic double bond (E/Z isomers).

In a second step, these formates are usually saponified to give the corresponding alcohols. Besides bisabolenes (as dehydration products), the mixture obtained in this way comprises, as main component, alpha-bisabolol and also farnesol. In order to obtain a clean product, the secondary component farnesol has to be separated off by distillation. Due to the similarity of the boiling points of the two components (bp. at 1 mbar: bisabolol: 110° C.; farnesol: 117° C.), this separation is extraordinarily technically complex. In addition, it is not really possible to cut farnesol fractions in a grade which would permit recycling to the bisabolol process.

The object was therefore to develop a process which allows farnesol-free or low-farnesol alpha-bisabolol to be provided in a processing and economically advantageous way from mixtures comprising alpha-bisabolol and farnesol.

Farnesol and its derivatives are likewise desired substances of value. Within the context of this invention, derivatives of farnesol are to be understood as meaning an ester of the formula (IX) with the definition of radicals given below. Thus, farnesol acetate of the formula (IX) where R³═CH₃ is a nature-identical product and has been detected in numerous essential oils. It is a desired specialty in the fragrance and aroma industry, where it is used in numerous compositions, in particular in green, herbal compositions, but also in castoreum and rose compounds. The most important scent aspects green-floral-rose-like are very desired in the fragrance industry. Here, farnesol acetate can be used in the range from 1% to 25%, in particular 3% to 8%.

According to the prior art, farnesol acetate is prepared by acetylation of farnesol of the formula (II) (see inter alfa Tetrahedron 1987, 5499; Chem. Commun. 2003, 1546; J. Org. Chem. USSR (Engl. Transl.) 1992, 1057; Synth. Commun. 1998, 2001). Other methods lead to farnesol acetate of the formula (XIV) by prenylation of precursors of the structures of the formulae (XI) or (XII) with (3-methylbut-2-enyl)magnesium chloride of the formula (XIII) (Synthesis 1991, 1130).

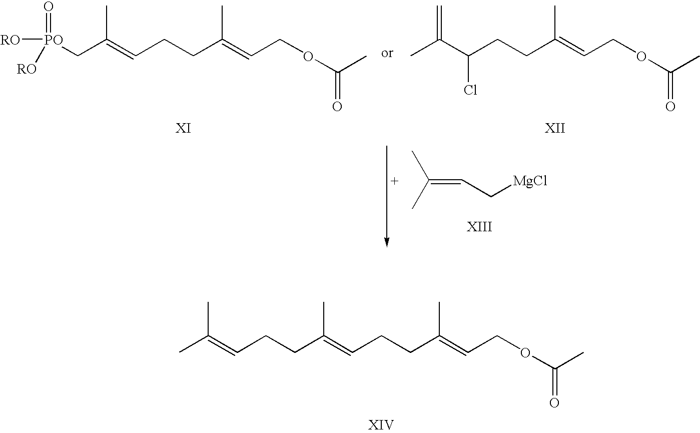

These processes require starting substances prepared which themselves have to be in in multistage methods.

The object was thus furthermore to commercially exploit the farnesol which is formed as coproduct, if appropriate in derivatized form.

### DESCRIPTION OF THE INVENTION AND THE PREFERRED EMBODIMENTS

The object was achieved through the provision of a method of producing farnesol-free or low-farnesol bisabolol of the formula (1)

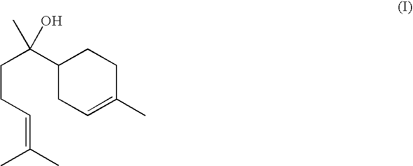

by treating mixtures comprising bisabolol of the formula (1) and farnesol of the formula (II)

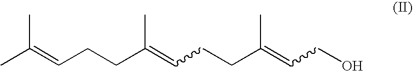

comprising the steps
a) reaction of the mixture with an at least equimolar amount, based on the amount of farnesol of the formula (II) used, of an ester of the formula (Ill)

R¹C(O)OR² (III),
where
R¹ is a straight-chain, branched or completely or partially cyclic, saturated or completely or partially unsaturated and/or aromatic and optionally substituted hydrocarbon radical having 1 to 12 carbon atoms and
R² is a C₁- to C₆-alkyl radical,
in the presence of a catalytic amount of an alkali metal and/or alkaline earth metal alkoxide having 1 to 6 carbon atoms, with selective formation of a farnesol ester of the formula (IV)

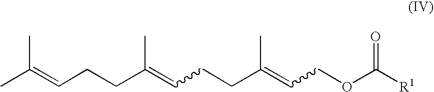

and an alcohol of the formula R²OH and with distillative separation of the alcohol of the formula R²OH formed, and of the ester of the formula (III) used in excess, if appropriate, and
b) distillative separation of the bisabolol used from the ester of the formula (IV) formed in step a).

The method according to the invention is suitable for producing farnesol-free or low-farnesol bisabolol of the formula (I)

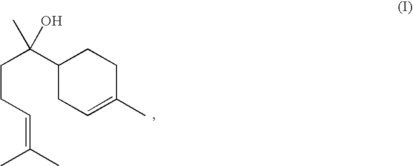

which is usually produced in the form of a diastereomer mixture of the formula (la) as described above with regard to the two stereogenic centers of the molecule in racemic form. The term farnesol-free bisabolol is understood here as meaning bisabolol or mixtures comprising bisabolol which bisabolol, besides any further components or impurities present, has a farnesol content of up to about 0.2% by weight, preferably of up to about 0.1% by weight, based on the total amount of the bisabolol or bisabol-containing mixture. The term low-farnesol bisabolol is understood here as meaning bisabolol or mixtures comprising bisabolol which bisabolol, besides any further components or impurities present, has a farnesol content of from about 0.2 to about 10% by weight, preferably from about 0.2 to about 5% by weight and particularly preferably from about 0.2 to about 3% by weight and very particularly preferably from about 0.2 to about 1% by weight, based on the total amount of the bisabolol or bisabolol-containing mixture.

The bisabolol of the formula (I) which can be produced according to the invention is produced, in the course of the method according to the invention, usually also with regard to further components and/or impurities apart from the farnesol, in purified form, often in a form contaminated only by low-boiling components which can easily be separated off.

Suitable starting materials for carrying out the method according to the invention are mixtures which comprise bisabolol of the formula (I) and farnesol of the formula (II)

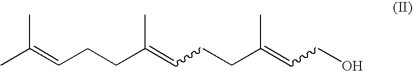

where the wavy lines refer in each case to E/Z mixtures with regard to the ethylenic double bonds. Mixtures to be used with preference as starting materials are those which consist of about 70 to about 99.9% by weight, preferably about 80 to about 99% by weight, of bisabolol and farnesol as the two main components. Possible further components may, for example, be solvents or byproducts from the preparation of the particular starting materials.

The method according to the invention comprises, within the scope of one embodiment, the steps a) and b), where in step a) the mixture used is reacted with an at least equimolar amount, based on the amount of used farnesol of the formula (II) present therein, of an ester of the formula (III)

R¹C(O)OR² (III).

The radical R¹ here is a straight-chain, branched or completely or partially cyclic, saturated or completely or partially unsaturated and/or aromatic and optionally substituted hydrocarbon radical having 1 to 12 carbon atoms. Preferably, R¹ is a C₆- to C₁₀-aryl radical, such as, for example, phenyl or naphthyl, preferably phenyl, which may be unsubstituted or can carry one or more, generally 1 to 3, identical or different substituents chosen from the group of the substituents C₁- to C₆-alkyl, halogen and C₁- to C₆-alkoxy. The radical R¹ is particularly preferably phenyl, ortho-methylphenyl, para-methylphenyl, ortho-para-dimethylphenyl, ortho-ortho-para-trimethylphenyl, ortho-methoxyphenyl or para-methoxyphenyl.

The radical R¹ can also be a straight-chain or branched or completely or partially cyclic C₁- to C₁₂-alkyl radical which can also carry one or more, generally 1 to 3, identical or different of the substituents as specified above and/or C₆- to C₁₀-aryl substituents. Here, C₁- to C₁₂-alkyl means C₁- to C₆-alkyl as described below and, moreover, for example heptyl, octyl, nonyl, decyl, undecyl or dodecyl. Preferred meanings of the radical R¹ are, for example: benzyl, straight-chain or branched decyl, such as, for example, the corresponding radicals of neodecanoic acids, or the radicals of the acids known under the tradename Versatic® Acid.

The radical R² is a C₁- to C₆-alkyl radical, such as, for example: methyl, ethyl, propyl, 1-methylethyl, butyl, 1-methylpropyl, 2-methylpropyl, 1,1-dimethylethyl, pentyl, cyclopentyl, 1-methylbutyl, 2-methylbutyl, 3-methylbutyl, 2,2-dimethylpropyl, 1-ethylpropyl, hexyl, cyclohexyl, 1,1-dimethyl propyl, 1,2-dimethylpropyl, 1-methyl pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Preferably, the radical R² is C₁- to C₃-alkyl, such as methyl, ethyl, propyl, particularly preferably methyl.

The term halogen is understood as meaning fluorine, chlorine, bromine or iodine, preferably fluorine, chlorine or bromine.

Esters of the formula (III) preferred according to the invention are optionally substituted benzoic esters, preferably benzoic C₁- to C₃-alkyl esters. An ester of the formula (III) which is particularly preferred according to the invention is methyl benzoate.

The chosen ester of the formula (III) is used in least equimolar amount, normally in an amount from 1 to about 3, preferably 1 to about 2, particularly preferably about 1.05 to about 1.7, equivalents, based on the amount of farnesol of the formula (II) present in the mixture used.

It has proven to be advantageous to use an ester of the formula (III) which has a higher boiling point than the corresponding alcohol R²OH or the C₁- to C₆-alkanol used.

The reaction according to step a) takes place in the presence of a catalytic amount of an alkali metal and/or alkaline earth metal alkoxide having 1 to 6 carbon atoms, with selective formation of a farnesol ester of the formula (IV)

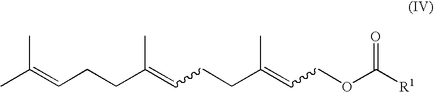

and of an alcohol of the formula R²OH, where the radicals R¹ and R² have the same meaning as in formula (III), Alkoxides to be used with preference which may be mentioned here are the lithium, sodium, potassium or calcium alkoxides of methanol, ethanol or n-propanol. Alkoxides preferred according to the invention are sodium methoxide, sodium ethoxide and sodium propoxide, particularly preferably sodium methoxide.

The term catalytic amount is understood as meaning an amount of from about 0.05 to about 10 mol % of the chosen alkoxide, based on the amount of farnesol used. The sodium methoxide to be used as preferred alkoxide is preferably used in amounts of from about 0.5 to about 10 mol %, preferably about 1.5 to about 7 mol %, based on the amount of farnesol used. For practical reasons, sodium methoxide is preferably used in the form of a methanolic solution.

During the reaction according to step a) of this embodiment of the method according to the invention, the alcohol of the formula R²OH formed by transesterification, and the ester of the formula (III) used in excess, if appropriate, are separated off from the resulting reaction mixture by distillation.

In the case of methyl esters, heating is in practice carried out to a bottom temperature of from about 70 to about 140° C., preferably about 80 to about 100° C. In this connection, it is advantageous, but not an essential part of the method according to the invention, to assist the distilling off of the alcohol by: applying a vacuum to about 5 mbar, passing through an inert stripping gas, preferably nitrogen and/or addition of an inert entrainer solvent such as, for example, heptane, toluene or xylene. This achieves a conversion of farnesol to the corresponding ester of the formula (IV), preferably to the farnesol benzoate, of more than 99% of theory.

According to step b) of this embodiment of the method according to the invention, the bisabolol used is separated off in farnesol-free or low-farnesol form from the ester of the formula (IV) formed in step a) by distillation. The distillation is advantageously carried out under a high vacuum, i.e. at pressures of up to 1 mbar, in which case, following the extraction of any higher-boiling impurities which may be present, bisabolol is obtained in the desired grade.

According to a further embodiment of the method according to the invention, the starting materials used are mixtures which comprise bisabolol formate of the formula (V)

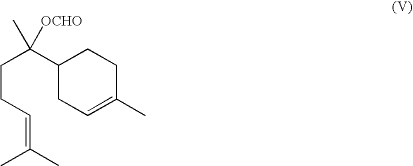

and farnesol formate of the formula (VI)

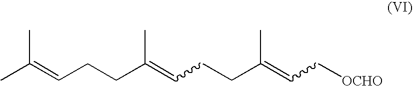

Mixtures of this type are preferably those which consist of about 70 to about 99.9% by weight, preferably about 80 to about 99% by weight, of bisabolol formate of the formula (V) and farnesol formate of the formula (VI) as the two main components. Possible further components may, for example, be solvents or byproducts from the preparation of the respective starting materials.

Firstly the free alcohols of the formulae (I) and (II) are liberated from said mixtures of the formates by an additional step.

According to the additional step of this embodiment of the method according to the invention, the mixture comprising the formates of the formulae (V) and (VI) is reacted with an at least equimolar amount, based on the total amount of the formates used, of a C₁- to C₆-alkanol in the presence of a catalytic amount of an alkali metal or alkaline earth metal alkoxide having 1 to 6 carbon atoms with the formation of the compounds of the formula (I) and (II) and a formic C₁- to C₆-alkyl ester. The formic C₁- to C₆-alkyl ester formed and also the C₁- to C₆-alkanol used in excess, if appropriate, are in the meantime removed from the reaction mixture formed by distillation to give a mixture comprising bisabolol of the formula (I) and farnesol of the formula (II).

The chosen C₁- to C₆-alkanol, preferably methanol, is used in the mixture of the starting materials in at least equimolar amount, usually in an excess of from about 1.05 to about 1.5, preferably about 1.05 to about 1.3, equivalents, based on the total amount of the formates used. Under the action of the catalytic amount, as described above, of the alkali metal or alkaline earth metal alkoxide used, preferably an alkoxide of the C₁- to C₆-alkanol used in this stage, particularly preferably sodium methoxide, transesterification gives the free alcohols of the formulae (I) and (II), and also the respective formic C₁- to C₆-alkyl esters, preferably methyl formate. The reaction is advantageously carried out at a temperature of from about 60 to about 90° C., where the formed formic C₁- to C₆-alkyl ester, preferably the formed methyl formate, and the C₁- to C₆-alkanol used in excess, if appropriate, are distilled off from the resulting reaction mixture.

The temperature required for this depends on the boiling point of the particular formic ester. In the case of methyl formate, heating is advantageously carried out at atmospheric pressure to about 60 to about 90° C., preferably to about 70 to about 80° C. In the case of longer-chain alcohols, the distilling off of the formic ester can also be aided by applying a vacuum or stripping with an inert gas, preferably nitrogen.

This gives, as residue, a mixture comprising bisabolol of the formula (I) and farnesol of the formula (II) which can be further reacted according to stages a) and b) of the embodiment of the method according to the invention described at the start.

Within the scope of a preferred embodiment, the method according to the invention for producing farnesol-free or low-farnesol bisabolol starting from mixtures comprising bisabolol formate of the formula (V) and farnesol formate of the formula (VI) can advantageously also be carried out so that the reactions passed through in the course of the reaction steps described above are passed through in one stage, with the base-catalyzed transesterification reactions, which are in equilibrium, proceeding alongside one another.

Accordingly, the present invention also relates to a method of producing farnesol-free or low-farnesol bisabolol of the formula (I) starting from mixtures comprising bisabolol formate of the formula (V)

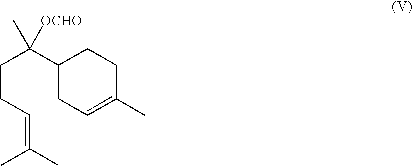

and farnesol formate of the formula (VI)

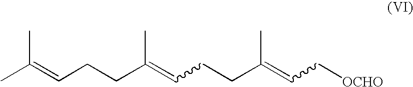

comprising the steps:
i) reaction of a mixture comprising the formates of the formulae (V) and (VI) with an at least equimolar amount, based on the amount of formate of the formula (V) used, of a C₁- to C₆-alkanol and with an at least equimolar amount, based on the amount of formate of the formula (VI) used, of an ester of the formula (III), where the radicals R¹ and R² can have the meanings given above, in the presence of a catalytic amount of an alkali metal or alkaline earth metal alkoxide having 1 to 6 carbon atoms, with the formation of bisabolol of the formula (I), of an ester of the formula (IV), and of a formic C₁- to C₆-alkyl ester and with distillative separation of the C₁- to C₆-alkanol used in excess, if appropriate, and of the formic C₁- to C₆-alkyl ester formed and
ii) distillative separation of the bisabolol of the formula (I) formed in step i) from the ester of the formula (IV).

According to step 1) of this embodiment of the present invention, the mixture comprising the formates of the formulae (V) and (VI) is reacted with an at least equimolar amount, based on the amount of bisabolol formate of the formula (V) used, of a C₁- to C₆-alkanol and with an at least equimolar amount, based on the amount of formate of the formula (VI) used, of an ester of the formula (III), where the radicals R¹ and R² can have the meanings specified above, in the presence of a catalytic amount of an alkali metal or alkaline earth metal alkoxide, with the formation of bisabolol of the formula (I), of the ester of the formula (IV), and of a formic C₁- to C₆-alkyl ester.

The chosen C₁- to C₆-alkanol corresponds here preferably to the alcohol radical R² of the ester of the formula (III) used and is preferably methanol. The alcohol chosen in each case is used in an at least equimolar amount, based on the amount of bisabolol formate of the formula (V) used present in the starting mixture. Preference is given to using the respective alcohol in an amount of from 1.05 to about 1.5 equivalents.

The chosen ester of the formula (III), preferably methyl benzoate, is used in at least equimolar amount, based on the amount of the farnesol formate of the formula (VI) used present in the starting mixture. Preference is given to using the respective ester in an amount of from1.05 to about 2 equivalents, particularly preferably from about 1.05 to about 1.7 equivalents.

Moreover, a catalytic amount of an alkali metal or alkaline earth metal alkoxide having 1 to 6 carbon atoms as described above is added to the reaction. For the purposes of this embodiment, the term catalytic amount is understood as meaning an amount of from about 0.05 to about 5 mol % of the chosen alkoxide, based on the amount of formates of the formulae (V) and (VI) used. The sodium methoxide to be used as preferred alkoxide is preferably used in amounts of from about 0.5 to about 3 mol %, particularly preferably about 1.5 to about 5 mol %, based on the amount of formates of the formulae (V) and (VI) used. For practical reasons, preference is given to using sodium methoxide in the form of a methanolic solution.

In the course of this embodiment, the particular C₁- to C₆-alkanol used, specifically methanol, and also the formed formic C₁- to C₆-alkyl ester, specifically methyl formate, are also distilled off from the resulting reaction mixture. The temperature required for this depends on the boiling point of the particular formic ester. In the case of methyl formate, heating is advantageously carried out at atmospheric pressure to about 60 to about 90° C., preferably to about 70 to about 80° C. In the case of longer-chain alcohols, the distilling off of the formic ester can also be assisted by applying a vacuum or stripping with an inert gas, preferably nitrogen. To aid the distillation operation, the measures specified above at the appropriate point are recommended.

From the residue obtained in step i), the formed bisabolol of the formula (I) is then separated off according to step ii) by distillation from the ester of the formula (IV) and thus obtained in the desired farnesol-free or low-farnesol form.

Due to the high reaction rates of this transesterification cascade, the overall process including the distillation of bisabolol can, if desired, also be carried out continuously, for example in an evaporation apparatus or a column.

The farnesol ester of the formula (IV) remaining in the distillation bottom can be saponified under aqueous-alkaline conditions by standard methods known per se to the person skilled in the art, and the farnesol recovered in this way can be reused. Alternatively, the formed ester of the formula (IV) can, after the bisabolol of the formula (I) has been separated off, be transesterified in the presence of an alcohol R²OH, for example under acid- or base-catalyzed conditions, and the ester of the formula (III) formed in the process can be returned to the method according to the invention,

The distillation bottom of process steps b) or ii) comprises usually about 70 to 90% by weight of esters of the formula (IV). It can be subjected to the following method according to the invention: a) the formed ester of the formula (IV) is reacted, after separating off the bisabolol of the formula (I), in the presence of a catalytic amount of an alkali metal or alkaline earth metal alkoxide with an ester of the formula (VIII)

R³C(O)OR⁴ (VIII).

After the reaction, β) the catalyst can be neutralized by adding an at least equimolar amount of a weak acid. If required, γ) the ester of the formula (IX) can be purified.

Preferably, the method involves the process steps α, βand γ.

The radicals can be chosen here such that the ester of the formula (IX) has a lower boiling point than the ester of the formula (IV). Preferably, the ester of the formula (IX) has a lower boiling point that the ester of the formula (IV).

The reaction equation below depicts the reaction diagrammatically. Besides the compounds listed, other compounds, in particular other esters, may, if appropriate, also be present in the reaction mixture.

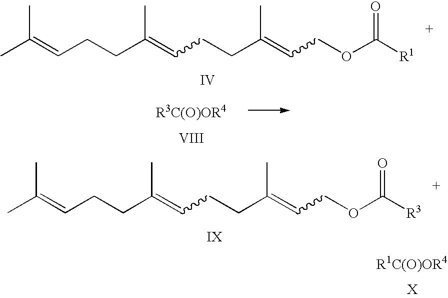

Alkoxides to be used with preference that may be mentioned here are the lithium, sodium, potassium or calcium alkoxides of methanol, ethanol or n-propanol. Alkoxides preferred according to the invention are sodium methoxide, sodium ethanoxide and sodium propoxide, particularly preferably sodium methoxide.

The alkoxides are preferably used in the form of a solution in the corresponding alcohol. For example, sodium methoxide can be used in the form of a 30% strength solution in methanol.

The term catalytic amount is to be understood as meaning an amount of from 0.05 to 7 mol %, preferably 1 to 5 mol %, of the chosen alkoxide, based on the amount of ester of the formula (IV) used. The sodium methoxide to be used as preferred alkoxide is preferably used in amounts of from 0.05 to 7 mol %, particularly preferably 1,5 to 7 mol %, based on the amount of ester of the formula (IV) used. For practical reasons, preference is given to using sodium methoxide in the form of a methanolic solution.

According to the invention, the transesterification reagent used is an ester of the formula (VIII)

R³C(O)OR⁴ (VIII),

Here, the definition of the radical R³ is the same as for R¹, and the definition for the radical R⁴ is the same as for R², with the proviso that R¹ and R³ are different. R⁴ can be chosen such that it is the same as R². However, it is also possible for R⁴ to be chosen such that it is not the same as R².

Preferably, R³ is chosen such that the resulting ester of the formula (IX) is a nature-identical compound. Thus, for example, esters of the formula (IX) where R³═CH₃, CH₂CH₃, (CH₂)₂CH₃, CH₂CH(CH₃)CH₃, (CH₂)₄CH₃, (CH₂)₅CH₃, (CH₂)₆CH₃, (CH₂)₇CH₃, (CH₂)₈CH₃, are known as pheromones (J. Appl. Entomol. 1996, 120, 463-466).

The term halogen is to be understood as meaning fluorine, chlorine, bromine or iodine, preferably fluorine, chlorine or bromine.

Esters of the formula (VIII) preferred according to the invention are methyl acetate, ethyl acetate, n-propyl acetate and isopropyl acetate. Particular preference is given to methyl acetate.

The chosen ester of the formula (VIII) is preferably used in an at least equimolar amount, normally in an amount of from 1 to 30, preferably 5 to 20, particularly preferably 10 to 15, equivalents, based on the amount of the ester of the formula (IV) present in the mixture used.

Since any excess esters of the formula (VIII) and the resulting esters of the formula (X) are preferably separated off from the ester of the formula (IX), it makes sense to choose the radicals such that the ester of the formula (VIII) and the ester of the formula (X) have a lower boiling point than the ester of the formula (IX). The ester of the formula (VIII) can here have a boiling point which is above or below the boiling point of the ester of the formula (X) or is the same as it. Preferably, the boiling point of the ester (VIII) is below the boiling point of the ester (X).

Furthermore, the boiling points of the esters of the formula (VIII), of the esters of the formula (X) and the esters of the formula (IX) are preferably sufficiently far apart in order to be able to obtain the respective esters in pure form during distillative purification.

Within the context of this application, where boiling points of compounds are compared with one another, then the boiling points of the pure substances which have been determined at the same pressure are compared. This pressure is normally atmospheric pressure, but may be higher or lower; in the case of decomposable substances, the boiling point is usually determined at pressures which are lower than atmospheric pressure.

The reaction according to step a) takes place with the formation of a farnesol ester of the formula (IX)

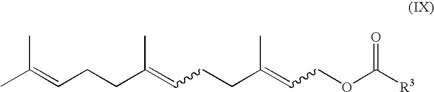

and of an ester of the formula (X)

R¹C(O)OR⁴ (X),

where the radical R¹ has the same meaning as in formula (III) and the radicals R³ and R⁴ have the same meaning as in formula (VIII).

The esters of the formula (IV) used are any desired mixtures of E/Z isomers. However, the esters of the formula (IV) may also be used in isomerically pure form.

Preferably, the abovementioned isomer mixtures of the formula (IV) give, through the reaction according to the invention, esters of the formula (IX) as a mixture of a corresponding isomer composition.

The radicals are preferably chosen such that the ester of the formula (IX) has a lower boiling point than the ester of the formula (IV).

Since the bottom produced during the bisabolol distillation has, besides the ester of the formula (IV), further unidentified secondary components, it is surprising that it is possible in a very simple way to obtain from it the ester of the formula (IX) in good yield and purity.

The distillation bottom of process steps b) or ii) does not necessarily have to serve as starting material. Instead, mixtures comprising esters of the formula (IV) can generally be used. If mixtures comprising esters of the formula (IV) which are not a distillation bottom of process steps b) or ii) are used, the method preferably involves process steps α, β and γ.

The reaction is generally carried out at temperatures and pressures such that the equilibrium shifts as completely as possible to the side of the esters of the formula (IX). The reaction can preferably be carried out at a reaction temperature from ambient temperature to reflux of the reaction mixture; preference is given to working at slightly elevated temperature, in the range from about 40 to 80° C., particularly preferably at about 50 to 60° C. The progress of the reaction can be monitored, for example, by means of thin-layer or gas chromatography.

When the reaction is complete, the catalyst is neutralized by adding an at least equimolar amount, based on the amount of catalyst used, of a weak acid. Preferably, the catalyst is neutralized by adding at least double the equimolar amount, based on the amount of catalyst used, of a weak acid.

According to the invention, weak acids have a pKₐ of b2 or more, preferably of 3 or more, particular preferably of 4 or more. The pKₐ is the negative of the base ten logarithm of the acid constant determined in water.

Suitable acids are in particular organic acids, preferably alkanecarboxylic acids, particularly preferably acetic acid. Adding a slight excess of this acid (or catalyst) gives a buffer with a pH of about 5 (in the aqueous extract). This “quenched” reaction mixture can be fractionally distilled without aqueous work-up to isolate the product of value. In this connection, the transesterification reagent used in excess can be obtained in pure form and returned to the process.

Preferably, the purification in process step γ) takes place by fractional distillation. The fractional distillation can be carried out as rectification. However, according to the invention, it is also possible to use other purification methods known to the person skilled in the art, such as, for example, dissolution and precipitation, adsorption methods and chromatography methods, electrophoresis, melting, in particular zone melting methods, freezing, standard solidification, crystallization, sublimation, growth methods or other transport reactions.

According to one embodiment of the method according to the invention, in process step γ),
γ1) the ester of the formula (VIII) is, if appropriate, in excess;
γ2) the ester of the formula (X);
γ3) the ester of the formula (IX)
   are fractionally distilled.

The radicals of ester of the formula (VIII), of the ester of the formula (X) and of the ester of the formula (IX) are preferably chosen here such that the boiling temperatures of the individual compounds to be separated are sufficiently different.

In a mixture to be separated according to the invention, for example, at the start of the distillation, methyl esters of the formula (VIII) where R⁴═CH₃, benzoic acid esters of the formula (X) where R¹=phenyl and esters of the formula (IX) where R³═CH₃ may be present alongside one another.

Preferably, the distillation cuts are chosen such that the compounds are produced in pure form, i.e. preferably have a purity of 90% GC or more, particularly preferably 95% GC or more.

It may be advantageous to reduce the pressure during the distillation. Here, the starting pressure may, for example, be the ambient pressure. Here, the pressure can be reduced by measures known to the person skilled in the art, for example by applying a vacuum.

Pressure and temperature are adjusted during the distillation such that fractional separation of the compounds can take place.

If appropriate, the distillation is furthermore assisted by pressing through an inert stripping gas, preferably nitrogen and/or adding an inert entrainer solvent, for example heptane, toluene or xylene.

The distilled-off ester of the formula (VIII) can be returned to the process at a suitable point, for example in process step α).

The distilled-off ester of the formula (X) can be returned to the process at a suitable point, for example in process steps a) or i) if it satisfies the criteria specified therein for an ester of the formula (III). In this connection, the returned ester of the formula (X) and the ester of the formula (III) used in the process are not necessarily the same, i.e. their radicals R² and R⁴ may be different.

In a preferred embodiment, the ester of the formula (X) and the ester of the formula (III) are identical, particularly preferably the ester of the formula (X) and the ester of the formula (III) are both methyl benzoate, and the ester of the formula (X) is returned to the process as ester of the formula (III).

The method according to the invention opens up a particularly advantageous access in economic and processing terms to pure or enriched bisabolol starting from readily accessible mixtures of bisabolol formate and farnesol formate. Surprisingly, it is possible here, in just one process step, to cleave the formates used to give the free alcohols and to convert farnesol completely selectively into a higher-boiling ester of the formula (IV). As a result, the hitherto still required saponification of the formates to be used, which includes an aqueous work-up including phase separation, can be saved. Through particularly simple distillation in terms of processing, bisabolol can be separated off from the reaction mixture, with no further work-up steps being required. Moreover, the farnesol ester remaining in the distillation bottom can be cleaved to give farnesol by simple ester saponification as explained or can be converted into a farnesol derivative by transesterification. This farnesol can in turn be converted into bisabolol according to the prior art, which renders the overall process very economic and resource-saving.

### Examples

The following experimental examples illustrate the method according to the invention without limiting it in any way:

GC method: Separation column 30 m DB-WAX/internal diameter 0.25 mm; film thickness 0.25 micrometer; starting temperature 120° C.; end temperature 250° C.; heating rate 5 K/min; detection: FID.

### Example 1

192 g of a mixture of the crude formates (V) and (VI), which comprised 46.0% bisaboloi formate (V) and 37.6% farnesol formate (VI) (content determination in each case by means of GC area %), was admixed with 20 g of methanol. 2.8 g of a 30% strength by weight methanolic sodium methoxide solution was then added; it was after-stirred for 30 min at ambient temperature. The reaction mixture was then heated to 80° C., during which methyl formate which formed was distilled off via a distillation bridge. At the bottom temperature of 80° C., 50.1 g of methyl benzoate were then added. A gentle stream of nitrogen was then passed through the reaction mixture via a gas inlet tube. After 4 hours, a GC sample revealed a composition of the reaction mixture of 40.6% bisabolol and 42.1% farnesol benzoate (in each case GC area %). Free farnesol was not detected.

Through direct distillation of the reaction mixture under a high vacuum (≦1 mbar) over a simple distillation bridge up to 119° C. transition, 105.3 g of distillate passed with a content of bisabolol of 70.1% (this corresponds to a yield of 94.2% based on bisabolol formate). The distillate comprised 0.3% farnesol. The bottom residue of 122.3 g had a content of farnesol benzoate of 76.2%.

### Example 2

The farnesol benzoate residue from Example 1 (122 g; 76.2% strength) was admixed at room temperature with 180 g of a 10% strength by weight solution of potassium hydroxide in methanol. The mixture was heated to reflux. Following after-stirring for one hour under reflux, 300 ml of water and 100 ml of toluene were added for work-up. The aqueous lower phase was separated off, and the organic phase was washed four times with 150 ml of water in each case until neutral. The organic phase was then concentrated on a rotary evaporator at 60° C. up to 15 mbar. This gave, as evaporation residue, 86.4 g of farnesol as EIZ isomer mixture with a purity of 72.2%.

### Example 3

192 g of a mixture of the crude formates (V) and (VI), which comprised 46.0% bisabolol formate (V) and 37.6% farnesol formate (VI) (according to GC area %), were initially introduced at 60° C. Then, 50.1 g of methyl benzoate, 13 g of methanol and 2.8 g of a 30% strength by weight sodium methoxide solution were added. While introducing a gentle stream of nitrogen by means of a gas inlet tube, the temperature of the reaction mixture was heated to 120° C. According to GC analysis, the mixture then comprised 41.94% bisabolol, 0.38% farnesol and 43.1% farnesol benzoate. Through distillation under a vacuum 1 mbar to 134° C. transition), bisabolol and other readily boiling secondary components were distilled off. As distillate, 103.5 g with a content of bisabolol of 69.0% and of farnesol of 0.87% were collected. This corresponds to a bisabolol yield of 91.1%, based on bisabolol formate used. In the distillation bottom, 110 g of farnesol benzoate with a content according to GC of 85.2% remained.

### Example 4

150 g of farnesol benzoate of the formula (IV) where R¹=phenyl (78.1% strength; =0.36 mol) were admixed at ambient temperature with 970 mg (18 mmol) of sodium methoxide. The mixture was heated to +50° C., and 340 g (4.60 mol) of methyl acetate of the formula (VIII) where R³═R⁴═CH₃ were allowed to run in. The mixture was stirred for 3 h at +50° C. and cooled to ambient temperature, and 2.16 g (36 mmol) of acetic acid were added. The mixture was then distilled. In the afore running, at atmospheric pressure to 100 mbar, 281 g of excess methyl acetate with a purity of >99% (according to GC) passed over. In the middle running (1 mbar/41-43° C. transition temperature), 44.4 g of methyl benzoate of the formula (X) were obtained where R¹=phenyl and R⁴═CH₃ with a purity of 99.0%. The main fraction of the product of value farnesol acetate of the formula (IX) where R³═CH₃ distilled at 0.5-1 mbar and a transition temperature of 112-119° C.

71.3 g of farnesol acetate were obtained (this corresponds to a yield of 75% of theory) with a purity of 96.7% (according to GC).

1-14. (canceled)

15. A process for producing farnesol-free or low-farnesol bisabolol of formula (I) 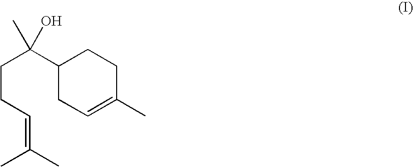
comprising
a) reacting a mixture which comprises said bisabolol of formula (I) and farnesol of formula (II) 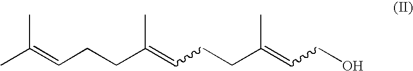
with an at least equimolar amount, based on the amount of farnesol of formula (II) used, of an ester of formula (III)
R¹C(O)OR² (III)
wherein
R¹ is a straight-chain, branched, or completely or partially cyclic, saturated or completely or partially unsaturated and/or aromatic and optionally substituted hydrocarbon radical having up to 12 carbon atoms; and
R² is a C₁- to C₆-alkyl radical;
in the presence of a catalytic amount of an alkali metal and/or alkaline earth metal alkoxide having up to 6 carbon atoms, resulting in selective formation of a farnesol ester of formula (IV) 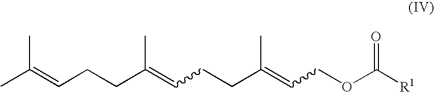
and an alcohol of formula R²OH;
b) distillatively separating said alcohol of formula R²OH and, optionally, excess ester of formula (III); and
c) distillatively separating said bisabolol from said ester of formula (IV).

16. The process of claim 15, wherein said mixture which comprises said bisabolol of formula (I) and said farnesol of formula (II) is prepared by a process comprising (1) reacting a mixture which comprises bisabolol formate of formula (V) 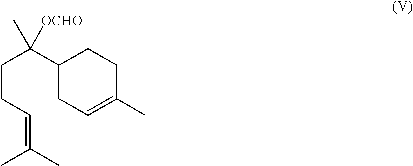
and farnesol formate of formula (VI) 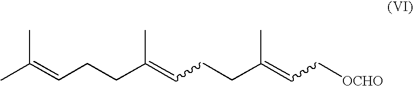
with an at least equimolar amount, based on the total amount of said formates, of a C₁- to C₆-alkanol in the presence of a catalytic amount of an alkali metal or alkaline earth metal alkoxide having 1 to 6 carbon atoms to form said compounds of formulae (I) and (II) and a formic C₁- to C₆-alkyl ester and (2) distillatively removing said formic C₁- to C₆-alkyl ester and, optionally, excess C₁- to C₆-alkanol.

17. A process for producing farnesol-free or low-farnesol bisabolol of formula (I) 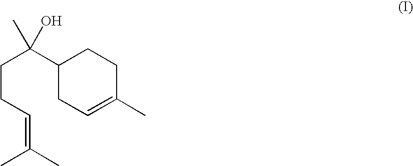
comprising
i) reacting a mixture which comprises starting bisabolol formate of formula (V) 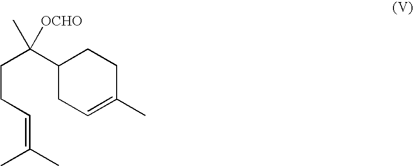
and farnesol formate of formula (VI) 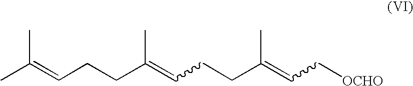
with an at least equimolar amount, based on the amount of formate of formula (V) used, of a C₁- to C₆-alkanol and with an at least equimolar amount, based on the amount of formate of formula (VI) used, of an ester of formula (III)
R¹C(O)OR² (III)
wherein
R¹ is a straight-chain, branched, or completely or partially cyclic, saturated or completely or partially unsaturated and/or aromatic and optionally substituted hydrocarbon radical having up to 12 carbon atoms; and
R² is a C₁- to C₆-alkyl radical;
in the presence of a catalytic amount of an alkali metal or alkaline earth metal alkoxide having 1 to 6 carbon atoms to form said bisabolol of the formula (I), an ester of formula (IV) 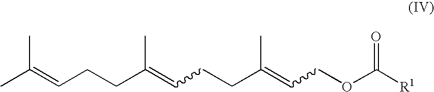
and a formic C₁- to C₆-alkyl ester;
ii) distillatively separating said formic C₁- to C₆-alkyl ester and, optionally, excess C₁- to C₆-alkanol; and
iii) distillatively separating said bisabolol from said ester of formula (IV).

18. The process of claim 15, wherein said ester of formula (III) has a higher boiling point than said alcohol of formula R²OH.

19. The process of claim 15, wherein said ester of formula (III) is an optionally substituted benzoic ester.

20. The process of claim 15, wherein said ester of formula (III) is methyl benzoate.

21. The process of claim 15, wherein said alkali metal alkoxide is sodium methoxide.

22. The process of claim 16, wherein said C₁- to C₆-alkanol is methanol.

23. The process of claim 15, wherein after c) said ester of formula (IV) is saponified under acidic or basic conditions to produce said farnesol of formula (II).

24. The process of claim 15, wherein after c) said ester of formula (IV) is transesterified in the presence of an alcohol of formula R²OH and the resulting ester of formula (III) is recycled into the process.

25. A process for producing farnesol esters of formula (IX) 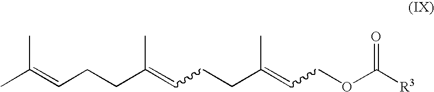
comprising
1) reacting mixtures which comprise esters of formula (IV) 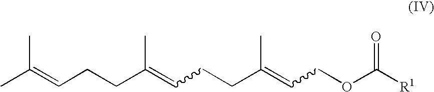
wherein
R¹ is a straight-chain, branched, or completely or partially cyclic, saturated or completely or partially unsaturated and/or aromatic and optionally substituted hydrocarbon radical having up to 12 carbon atoms;
with an ester of the formula (VIII)
R³C(O)OR⁴ (VIII)
wherein
R³ is a straight-chain, branched, or completely or partially cyclic, saturated or completely or partially unsaturated and/or aromatic and optionally substituted hydrocarbon radical having up to 12 carbon atoms; and
R⁴ is a C₁- to C₆-alkyl radical;
with the proviso that R¹ and R³ are different from one another
in the presence of a catalytic amount of an alkali metal or alkaline earth metal alkoxide;
2) neutralizing said alkali metal or alkaline earth metal alkoxide by adding an at least equimolar amount of a weak acid; and
3) purifying said ester of formula (IX).

26. The process of claim 15, further comprising reacting said ester of formula (IV) with an ester of the formula (VIII)
R³C(O)OR⁴ (VIII)
wherein
R³ is a straight-chain, branched, or completely or partially cyclic, saturated or completely or partially unsaturated and/or aromatic and optionally substituted hydrocarbon radical having up to 12 carbon atoms; and
R⁴ is a C₁- to C₆-alkyl radical;
with the proviso that R¹ and R³ are different from one another;
in the presence of a catalytic amount of an alkali metal or alkaline earth metal alkoxide, resulting in a farnesol ester of formula (IX)

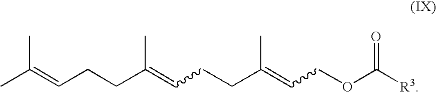

27. The method of claim 26, further comprising
1) neutralizing said alkali metal or alkaline earth metal alkoxide by adding an at least equimolar amount of a weak acid; and
2) purifying said ester of formula (IX).

28. The proses of claim 25, wherein said ester of formula (VIII) is methyl acetate.
